(19) United States
(12) Patent Application Publication
(10) Pub. No.: US 2019/0030125 A1
(43) Pub. Date: Jan. 31, 2019

(54) USE OF FIBULIN-5 FOR THE TREATMENT OF KELOID SCARS

(71) Applicant: Vessl Therapeutics Ltd, Haifa (IL)

(72) Inventor: Moshe Y. FLUGELMAN, Haifa (IL)

(21) Appl. No.: 15/533,691

(22) PCT Filed: Dec. 7, 2015
(86) PCT No.: PCT/IL2015/051187

§ 371 Date: Jun. 7, 2017

Related U.S. Application Data
(60) Provisional application No. 62/088,607, filed on Dec. 7, 2014.

(51) Int. Cl. A61K 38/17; A61K 31/721; A61P 17/02; A61K 9/00

(52) U.S. Cl. CPC A61K 38/1741; A61K 9/0019; A61P 17/02; A61K 31/721

(57) ABSTRACT

Use of fibulin-5 in the manufacture of a medicament for treating or preventing formation of a keloid scar or a medical condition associated therewith is provided herewith.

### FIELD AND BACKGROUND OF THE INVENTION

The present invention, in some embodiments thereof, relates to the use of fibulin-5 for the treatment of keloid scars.

A keloid scar is pathological tissue that appears after skin injury and invades beyond the original borders of the wound.^{1,2} Beyond the aesthetic issue, the keloid scar can limit the range of motion when it develops above a joint (contracture).³ Despite the relatively high prevalence of keloids in the general population, the mechanisms underlying their formation are only partially understood. This is reflected in the multiple treatment modalities, of which no single treatment has proven to be widely effective.^{3,4}

Keloids scars are characterized by excessive extracellular matrix (ECM) accumulation, including collagen I and collagen III, in their dermis and subcutis layers.^{5,6} Among the mechanisms that have been proposed for keloid formation and for the high proliferation rate of fibroblast-like cells (FLCs) isolated from keloids, are the elevated expression of certain cytokines, including transforming growth factor-b(TGF-beta^{3,7} and insulin-like growth factor-1 (IGF-1),^{4,8} and an imbalance between proliferation and apoptotic cell death.^{9,10}

Fibulin-5 is a glycoprotein secreted by many cell types, and is a component of the ECM. Fibulin-5 contains an RGD motif, enabling its binding to integrin proteins.¹¹ This binding enables the involvement of fibulin-5 in an intracellular signaling chain that affects fibroblast proliferation, migration and adherence.¹²⁻¹⁵ Overexpression of fibulin-5 was reported to promote in-vivo wound healing, by increasing the amount of granulation tissue.¹⁶ Fibulin 5 has an essential role in elastic fiber formation;^{17,18} however keloid scars are lacking in elastic fibers.^{19,20} Low levels of fibulin-5 were reported in keloid scars, perhaps due to the accumulation of chondroitin sulphate in the ECM of this tissue.²⁰ Integrin beta-1 was shown to mediate adhesion of smooth muscle cell to fibulin-5.¹⁵

Additional background art includes:

U.S. Pat. Appl. No. 20040126788

Davidson and Giro J Invest Dermatol. 2006 December; 126(12):2563-4

### SUMMARY OF THE INVENTION

According to an aspect of some embodiments of the present invention there is provided a method of treating a keloid scar or a medical condition associated therewith, the method comprising administering to a subject in need thereof a therapeutically effective amount of fibulin-5, thereby treating the keloid scar.

According to an aspect of some embodiments of the present invention there is provided a use of fibulin-5 in the manufacture of a medicament for treating or preventing formation of a keloid scar or a medical condition associated therewith.

According to an aspect of some embodiments of the present invention there is provided Fibulin-5 for the treatment or prevention of a keloid scar or a medical condition associated therewith.

According to an aspect of some embodiments of the present invention there is provided a method of inhibiting proliferation and/or adherence of fibroblast-like cells from a keloid scar, the method comprising contacting the fibroblast-like cells with an effective amount of fibulin-5, thereby inhibiting proliferation and/or adherence of fibroblast-like cells from the keloid scar.

According to some embodiments of the invention, the contacting is effected in-vivo.

According to some embodiments of the invention, the contacting is effected ex-vivo or in-vitro.

According to some embodiments of the invention, the fibroblast-like cells are comprised in a tissue.

According to some embodiments of the invention, the fibroblast-like cells are primary cells.

According to some embodiments of the invention, the fibroblast-like cells are a cell line.

According to an aspect of some embodiments of the present invention there is provided a cosmetic composition comprising an effective amount of fibulin-5 and a cosmetically acceptable carrier.

According to some embodiments of the invention, the keloid scar is caused by external injuries.

According to some embodiments of the invention, the keloid scar is caused by surgical procedures.

According to some embodiments of the invention, the medical condition is selected from the group consisting of is selected from the group consisting of pain, inflammation and vascularization.

According to some embodiments of the invention, the administering is effected locally.

According to some embodiments of the invention, the administering is effected epicutaneously, subcutaneously or intradermally.

According to some embodiments of the invention, the fibulin-5 comprises an integrin beta binding domain.

According to some embodiments of the invention, the fibulin-5 is full length fibulin-5.

According to some embodiments of the invention, wherein the fibulin-5 is human fibulin-5.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F show extracellular matrix (ECM) and collagen structures of a keloid section. FIG. 1A—Appearance of the epidermis in a keloid sample. High collagen deposits in the dermis layer can be seen (thick arrow). FIG. 1B—Masson trichrome stain showing large deposit of collagen in the dermis layer. FIG. 1C—Scar explants were cultured and fibroblast cells with spindle-shape were isolated from all the keloid scars. FIG. 1D—In petri dishes of cell lines E&F, the formation of extensions between the migrated cells was observed. FIG. 1E—Positive coomassie blue staining of cell-extensions shows protein composition of the structures. FIG. 1F—Negative DAPI staining, which shows the nucleus location of the cells that forms these extensions.

FIG. 2 shows proliferation of fibroblast like cells (FLCs) cultured on fibulin-5 or on gelatin-coated surface. Proliferation rates of several FLC lines were assayed for 72-96 hr. All cells were seeded into wells pre-coated with fibulin-5 or gelatin. A significant reduction (p<0.05) in cell-proliferation rates was found for all tested FLC-lines (left), each test consisted of 5 repetitions. Pooled samples from all proliferation experiments (N=50) showed a significant inhibition of FLC on fibulin-5 coating compared to gelatin (right). *P=0.002.

FIGS. 3A-B show the adhesion rates of keloid derived fibroblast-like cell (FLCs). FIG. 3A—Time-dependent adhesion of keloid derived fibroblast-like cells (FLC), in the presence of fibulin-5 or gelatin as a pre-coating. FLC F line cells were plated 2×10⁴ cells/well with five repeats for each coating treatment, fibulin-5 or gelatin. The cells were attached to the pre-coated surface and reached full adhesion after about 180 min. The wells were washed three times with PBS at time points of 20,40,60,90,120,180 and 240 min; and positive control wells were left unwashed. The amount of adherent cells was calculated using an XTT assay at the end of the entire experiment. FIG. 1B—Adherence of FLCs on fibulin-5 coated surfaces, with or without anti-human CD29 antibody. FLC F cells (2×10⁴/well) were seeded on fibulin-5 coated 90 6-well plates. The control group was treated with DMEM medium supplemented with 0.5% FBS at a final volume of 100 well. The treatment group was treated with the same medium with an addition of 20 μg/10⁶ cells of anti-human CD29 antibody. The wells were washed with PBS at time points of 90,120,180 minutes. The amount of adherent cells (%) was calculated using the XTT assay.

### DESCRIPTION OF SPECIFIC EMBODIMENTS OF THE INVENTION

The present invention, in some embodiments thereof, relates to the use of fibulin-5 for the treatment of keloid scars. Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

A keloid scar is a pathological tissue that appears after skin injury, and that is more aggressive than hypertrophic scars. Keloid scars are characterized by increased proliferation of fibroblast-like cells and the accumulation of extra-cellular matrix, mainly collagen. Fibulin-5, a glycoprotein secreted by many cell types, is a component of the extracellular matrix.

Whilst conceiving the present invention to practice, the present inventors investigated the effect of fibulin-5 on the adhesion and proliferation of fibroblast-like cells (FLCs) derived from keloid scars, and the role of integrin beta-1 in these activities.

Fibulin-5 treatment of keloid scars or cells derived there from revealed a yet unknown inhibitory role of the protein. Fibulin 5 is able to reduce proliferation of FLCs from keloid scars and reduce their surface adhesion. The effect was found to be mediated by integrin beta-1.

As is shown hereinbelow and in the Examples section which follows, fibroblast-like cells were isolated from six keloid scars and cultured on plates coated with fibulin-5 or with gelatin. Cells were incubated for 72-96 hours to examine proliferation rates; and incubated for 240 minutes, with washings at 20,40,60,90,120,180 minutes, to assess adhesion rates. To examine the role of integrin beta-1, the anti-human integrin beta-1(CD29) antibody was added to the culture medium.

The fibroblast-like cells cultured on a fibulin-5 coated surface showed a significantly reduced proliferation rate and a delayed adhesion rate, compared to cells cultured on gelatin coated dishes. Adherence of fibroblast-like cells to fibulin-5 pre-coated wells was significantly reduced in the presence of anti-human integrin beta-1 (CD29) antibodies.

Thus these findings suggest a pivotal role of fibulin-5 on the adhesion and proliferation of human keloid-derived cells, through binding to integrin beta-1 and place it as a therapy for this yet untreated medical condition.

Thus, according to an aspect of the invention, there is provided a method of inhibiting proliferation and/or adherence of fibroblast-like cells from a keloid scar, the method comprising contacting the fibroblast-like cells with an effective amount of fibulin-5, thereby inhibiting proliferation and/or adherence of fibroblast-like cells from the keloid scar.

As used herein the phrase “keloid scar” or “keloid” refers to the formation of a skin scar which, depending on its maturity, is composed mainly of either type III (early) or type I (late) collagen. It is a result of an overgrowth of granulation tissue (collagen type 3) at the site of a healed skin injury which is then slowly replaced by collagen type 1. Keloids are firm, rubbery lesions or shiny, fibrous nodules, and can vary from pink to the colour of the patient's flesh or red to dark brown in color. A keloid scar is benign and not contagious, but sometimes accompanied by severe itchiness, pain, and changes in texture. In severe cases, it can affect movement of skin. Keloid scars are seen 15 times more frequently in highly pigmented ethnic groups than in Caucasians.

Keloids should not be confused with hypertrophic scars, which are raised scars that do not grow beyond the boundaries of the original wound.

According to some embodiments of the invention, the keloid is caused by an external injury.

According to some specific embodiment, the keloid is a result of a surgical procedure.

More specifically, and yet according to some embodiments of the invention, the keloid is a result of a skin injury caused by acne, burns, chickenpox, ear piercing, scratches, surgical cuts or vaccination sites.

As used herein the phrase “fibroblast-like cell” or FLCs refers to a type of cell that synthesizes the extracellular matrix and collagen, the structural framework (stroma) for animal tissues, and plays a critical role in wound healing. Fibroblasts are the most common cells of connective tissue in animals. Morphologically, the cells (FLCs and fibroblasts) are large, flat, elongated (spindle-shaped) cells possessing processes extending out from the ends of the cell body. The cell nucleus is flat and oval. The cells produce tropocollagen, which is the forerunner of collagen, and ground substance, an amorphous gel-like matrix that fills the spaces between cells and fibres in connective tissue.

As used herein the term “adherence” refers to the adhesion rate of keloid-derived fibroblast-like cells.

Methods of monitoring cell adherence are well known in the art and are described in great details in Humphries Methods Mol Biol. 2009; 522:203-10, which is herein incorporated by reference. These include, but are not limited to, the attachment assay, which employs a colorimetric detection of bound cells, is based on Kueng et al. (Anal Biochem 182:16-19, 1989), which is herein incorporated by reference, and the spreading assay, which employs phase contrast microscopy to measure the flattening of adherent cells, is based on the method of Yamada and Kennedy (J Cell Biol 99:29-36, 1984), which is herein incorporated by reference. Another cell adhesion assay is described in Example 1 of the Examples section which follows.

As used herein the term “proliferation” refers to the increase in cell number as a result of cell growth and division.

Methods of monitoring cell proliferation are well known in the art and include, but are not limited to, manual cell counting, the MTT test which is based on the selective ability of living cells to reduce the yellow salt MTT (3-(4,5-dimethylthiazolyl-2)-2,5-diphenyltetrazolium bromide) (Sigma, Aldrich St Louis, Mo., USA to a purple-blue insoluble formazan precipitate; the thymidine incorporation assay, the BrDu assay [Cell Proliferation ELISA BrdU colorimetric kit (Roche, Mannheim, Germany]; the TUNEL assay [Roche, Mannheim, Germany]; the Annexin V assay [ApoAlert® Annexin V Apoptosis Kit (Clontech Laboratories, Inc., CA, USA)]; the Senescence associated-β-galactosidase assay (Dimri G P, Lee X, et al. 1995.

As used herein the term “inhibit” refers to a decrease of at least 5%, 10%, 15%, 20%, 30%, 40%, 50%, 60%, 70%, 80%, 90%, 100% and even more in cell proliferation and/or adherence in the presence of the compound e.g., Fibulin-5, as compared to control cells not treated with Fibulin-5, or treated with a control vehicle. According to some embodiments, the control is a duplicate cell sample of the same developmental stage and biological source.

As used herein, the term “Fibulin-5” refers to the expression product of FBLN5 gene. The protein encoded by this gene is a secreted, extracellular matrix protein containing an Arg-Gly-Asp (RGD) motif and calcium-binding EGF-like domains. The protein is an integrin-beta1 binding protein. It promotes adhesion of endothelial cells through interaction of integrins and the RGD motif. It plays a role in vascular development and remodeling. Defects in this gene are a cause of autosomal dominant cutis laxa, autosomal recessive cutis laxa type I (CL type I), and age-related macular degeneration type 3 (ARMD3).

Fibulin-5 is also referred to as, UP50, DANCE, ARCL1, FIBL5, ADCL2. ARMD3 and EVEC.

According to a specific embodiment, the Fibulin-5 is a human Fibulin-5 or any other non-immunogenic homolog thereof. For veterinary treatments other homologs of Fibulin-5 can be used, dependent on the intended use.

According to a specific embodiment, the Fibulin-5 is a truncated form although it still comprises an integrin-beta1 binding site at the N-terminus of the protein (RGD) See also, Yangisawa et al. J Cell Commun Signal. 2009 December; 3(3-4): 337-347.

According to a specific embodiment, the fibulin-5 is full-length fibulin-5. According to a specific embodiment, the Fibulin-5 is provided in GenBank Accession Number: FBLN5_HUMAN, Q9UBX5, NP_006320. The mature form is 448 amino acids long and provided in sequence SEQ ID NOs: 1 (or SEQ ID NO: 2 encoding same).

It will be appreciated that Fibulin-5 can be provided per se or conjugated to proteinaceous or non-proteinaceous moieties. Such embodiments are of particular value in in-vivo applications.

Exemplary non-proteinaceous moieties which may be used according to the present teachings include, but are not limited to, polyethylene glycol (PEG), Polyvinyl pyrrolidone (PVP), poly(styrene comaleic anhydride) (SMA), and divinyl ether and maleic anhydride copolymer (DIVEMA).

Such a molecule is highly stable (resistant to in-vivo proteolytic activity probably due to steric hindrance conferred by the non-proteinaceous moiety) and may be produced using common solid phase synthesis methods which are inexpensive and highly efficient, as further described hereinbelow. However, it will be appreciated that recombinant techniques may still be used, whereby the recombinant peptide product is subjected to in-vitro modification (e.g., PEGylation as further described hereinbelow).

Alternatively, or additionally the Fibulin-5 can be in frame fused to a proteinaceous moiety such as an immunoglobulin to improve its bioavailability.

It is noted that other relevant teachings for promoting the protein availability are described in WO2011/138785, which is hereby incorporated by reference in its entirety.

The term “peptide” or “protein” as used herein encompasses native peptides/polypeptide (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides/proteins of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids, the first can be added to increase bioavailability.

According to a specific embodiment, the fibroblast-like cells are contacted with the Fibulin-5 in-vivo.

According to a specific embodiment, the fibroblast-like cells are contacted with the Fibulin-5 in-vitro or ex-vivo.

According to the latter option, the cells can be comprised in the keloid (as part of a tissue) or they can be isolated cells e.g., forming a monolayer (with possible nodules) on a culture dish.

Accordingly, the cells can be a primary cell culture.

Alternatively, the cells are a cell line.

The cells are contacted with an effective amount of Fibulin-5 for a time period sufficient to inhibit proliferation/adherence.

The Figulin-5 protein (or an active peptide derived therefrom) can be provided per se or as part of a nucleic acid construct where the nucleic acid sequence encoding Fibulin-5 under a cis-acting regulatory element is ligated into a relevant expression vector.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

An active peptide of Fibulin-5 is selected having at least 80%, 90%, or 95% homology (e.g., identity) to wild-type Fibulin-5 as long as it is able to inhibit cell proliferation/adhesion, e.g., in an integrin beta1 dependent manner. According to a specific embodiment, the Fibulin-5 peptide comprises an integrin beta1 binding domain (found at the N-terminus of the protein including an RGD motif).

Human Fibulin-5 can thus be purified or synthesized using methods which are well known in the art. For example, recombinant DNA technology can be used to generate Fibulin-5 such as by the use of viral vectors e.g., retroviruses.

Alternatively or additionally the protein of Fibulin-5 (mature) is available from a plurality of vendors including Aviscera Bioscience.

As used herein the term “subject” refers to an individual having a keloid or being at risk of developing a keloid. According to a specific embodiment, the subject is undergoing or has undergone a surgical procedure.

According to a specific embodiment the subject is a mammal such as a human being, however veterinary used are also contemplated.

According to a specific embodiment, the subject is a juvenile since keloids are common in young people between the ages of 10 and 20.

According to a specific embodiment the subject is an African American, Asian or Hispanic which are more susceptible to keloids.

The ability of Fibulin-5 to inhibit Fibroblast-like cells proliferation/adhesion can be harnessed towards clinical applications.

Accordingly, there is provided a method of treating a keloid scar or a medical condition associated therewith, the method comprising administering to a subject in need thereof a therapeutically effective amount of fibulin-5, thereby treating the keloid scar.

Alternatively, there is provided a use of fibulin-5 in the manufacture of a medicament for treating or preventing formation of a keloid scar or a medical condition associated therewith.

Alternatively or additionally, there is provided a Fibulin-5 for the treatment or prevention of a keloid scar or a medical condition associated therewith.

As used herein the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein the term “preventing” refers to inhibiting the onset of the condition.

It is noted that ‘prophylactic treatment’ in regards keloid scar formation refers to the administration of the composition to a site at which an injury has recently occurred which is suspected of or at risk of leading to the formation of a keloid scar. Alternatively, the composition may be applied at a point at which keloid scar formation may have started at the molecular level but has not yet produced a visible scar or any visible signs of impending scar formation.

The Fibulin-5 (protein or nucleic acid sequence encoding same e.g., SEQ ID NOs: 1 and 2), hereinafter “Fibulin-5” of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the Fibulin-5 accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, enteral (e.g., oral) or paraenteral delivery.

According to a specific embodiment, the Fibulin-5 is administered in a local manner i.e., to the skin e.g., to the afflicted tissue region, i.e., keloid.

Methods of administering an active agent into a skin are known in the art and include, for example, intradermal injections, gels, liquid sprays and patches which comprise the active agent and which are applied on the outer surface of the skin.

According to some embodiments of the invention, administration of the active agent (Fibulin-5) into the skin of the subject is performed topically (on the skin).

According to some embodiments of the invention, administration of the active agent (Fibulin-5) into the skin of the subject is performed non-invasively, e.g., using a gel, a liquid spray or a patch comprising the active ingredient, which are applied onto the skin of the subject.

There are two main types of skin patches which can be used to administer the (Fibulin-5) into the skin of a subject. These are the reservoir type patch and the matrix type patch. The reservoir patch usually contains a structure filled with a solid drug (active agent) and a dilute solution, or a highly concentrated drug solution within a polymer matrix and is surrounded by a film or membrane of rate-controlling material. The matrix patch contains a drug and a polymer which form a homogenous system from which the drug is released by diffusion into the external environment. It should be noted that as the release continues, its rate in the matrix type patch usually decreases since the active agent has a progressively longer distance and therefore requires a longer diffusion time to release. For further details and examples of transdermal drug delivery see Prausnitz M R., et al., 2004. Nature Reviews, 3:115-124; Scheindlin S., 2004. Transdermal drug delivery: Past, present, future. Molecular Interventions. Vol. 4:308-312; Prausnitz M R and Langer R., 2008, Nature Biotechnology. 26:1261-1268; Tanner T, and Marks R, 2008, Delivery drugs by transdermal route: review and comment. Skin Research and Technology, 14: 249-260; each of which is hereby incorporated by reference in its entirety).

A non-limiting example of an epicutaneous drug delivery patch, which can be used to administer the Fibulin-5 into the skin according to the teachings of the invention, is described in Senti G., et al., 2009, J Allergy Clin Immunol. September 4. [Epub ahead of print], which is hereby incorporated by reference in its entirety).

According to some embodiments of the invention, administering the Fibulin-5 to the skin is performed using a reservoir type patch.

According to some embodiments of the invention, administering the Fibulin-5 to the skin is effected on an intact skin (e.g., a skin which has not been breached, peeled or physically/chemically permeabilized).

For example, administering into an intact skin can be performed using an occlusive patch with semi-solid reservoir and a plastic backing adhesive contour and protective removable cover.

A semi-solid reservoir can be any gel, cream, ointment, emulsion, suspension, microparticles, using various excipients such as fats, oils (e.g., mineral oil, Vaseline, vegetable oil or silicon oil), polymers, gelling agent, suspending agent, stabilizers, hydrophilic solvents, Propylene glycol, polyethylene glycols, stabilizing surfactants, colloids etc. and their combinations.

It should be noted that in order to increase delivery of the active agent into the skin, the active agent can be formulated with various vehicles designed to increase delivery to the epidermis or the dermis layers. Such vehicles include, but are not limited to liposomes, dendrimers, noisome, transfersome, microemulsion and solid lipid nanoparticles (for further details see Cevc, G. Transfersomes, liposomes and other lipid suspensions on the skin: permeation enhancement, vesicle penetration, and transdermal drug delivery. Crit. Rev. Ther. Drug Carrier Syst. 13, 257-388 (1996), which is hereby incorporated by reference in its entirety; Kogan A, Garti N. Microemulsions as transdermal drug delivery vehicles. Adv Colloid Interface Sci 2006; 123-126:369-385, which is hereby incorporated by reference in its entirety). In addition, the active agent can be mixed with chemical enhancers such as sulphoxides, azones, glycols, alkanols and terpenes which enhance delivery of active agents into the skin (for further details see Karande P, Jain A, Ergun K, Kispersky V, Mitragotri S. Design principles of chemical penetration enhancers for transdermal drug delivery. Proc Natl Acad Sci USA 2005; 102:4688-4693; Williams A C, Barry B W. Penetration enhancers. Adv Drug Deliv Rev 2004; 56:603-618; and Smith, E W.; Maibach, H I., editors. Boca Raton, Fla.: Taylor and Francis Group; 2006. Percutaneous Penetration Enhancers; each of which is hereby incorporated by reference in its entirety).

The patch may include the Fibulin-5 formulated within an emulsion designed to facilitate permeabilization of drugs to the epidermis or the dermis. For example, the patch may comprise the Fibulin-5 within an oil-in-glycerin emulsion, which is designed to facilitate permeabilization of the Fibulin-5 through the stratum-corneum and into the dermis. A non-limiting example of an oil-in-glycerin emulsion suitable for delivery through the stratum-corneum into the dermis is described in US Patent Application No. 20040067244, which is hereby incorporated by reference in its entirety. Such an oil-in-glycerin emulsion exhibits a mean droplet size below one micron, and comprises a continuous glycerin phase; at least one vegetable oil comprising an internal phase; at least one emulsifying stabilizer; and at least one bioactive compound comprising at least one hydrophobic, moiety within its structure, wherein the composition facilitates permeabilization of the bioactive compound through the stratum-corneum and into the dermis.

According to some embodiments of the invention, administering the Fibulin-5 to the skin is effected on a breached skin [e.g., a skin that has been permeabilized (e.g., ruptured) with an external object and the like].

According to some embodiments of the invention, breaching of the skin is effected temporarily (e.g., performed for a pre-determined short period) and is designed to enable better permeabilization of the active ingredient into the skin.

Breaching of the skin can be performed, for example, by introducing micro-holes (e.g., microchannels) in the outer layer of the skin. Such microchannels can be formed using for example, the Radio-Frequency (RF)-Microchannel™ (TransPharma Medical™ Ltd.) technology [Hypertext Transfer Protocol ://World Wide Web (dot) transpharma-medical (dot) com/technology_rf (dot) html].

Additionally or alternatively, delivery of the active agent (e.g., the Fibulin-5) from the patch to the epidermis layer of the skin can be enhanced using physical enhancers known in the art such as ultrasound, ionophoresis, electroporation, magnetophoresis, microneedle and continuous mixing [see e.g. Rizwan M, Aqil M, Talegaonkar S, Azeem A, Sultana Y, Ali A. Enhanced transdermal drug delivery techniques: an extensive review of patents. Recent Pat Drug Deliv. Formul. 2009; 3(2).105-24 which is here by incorporated by reference in its entirety].

According to some embodiments of the invention, administering the Fibulin-5 is performed by an intradermal injection.

The Fibulin-5 can be administered into the dermal layer of the skin of the subject by an intradermal injection as described for the Mantoux C (1908) test. Briefly, the Fibulin-5 can be injected intracutaneously (using for example, a 0.5-ml or 1.0 ml tuberculin syringe through a 26-gauge or 27-gauge needle). The syringe can be placed at an angle of 45 degrees to the skin, and the bevel of the needle is angled downward, facing the skin, and penetrating entirely but not deeper than the superficial layers of the skin. A volume of approximately 0.01 to 0.05 ml (e.g., about 0.02 ml) is gently injected to produce a small superficial bleb (Middleton's Allergy principles&practice, 6^{th} edition 2003).

According to some embodiments of the invention, administering the Fibulin-5 is performed using a liquid spray (e.g., a spray which includes the Fibulin-5 in a pre-determined concentration and dosage).

According to some embodiments of the invention, administering the Fibulin-5 is performed using a gel (e.g., a gel which includes the Fibulin-5 in a pre-determined concentration and dosage).

Implants useful in practicing the methods disclosed herein may be prepared by mixing a desired amount of a stabilized Fibulin-5 (such as non-reconstituted BOTOX®) into a solution of a suitable polymer dissolved in methylene chloride. The solution may be prepared at room temperature. The solution can then be transferred to a Petri dish and the methylene chloride evaporated in a vacuum desiccator. Depending upon the implant size desired and hence the amount of incorporated Fibulin-5, a suitable amount of the dried Fibulin-5 incorporating implant is compressed at about 8000 p.s.i. for 5 seconds or at 3000 p.s.i. for 17 seconds in a mold to form implant discs encapsulating the Fibulin-5. See e.g. Fung L. K. et al., Pharmacokinetics of Interstitial Delivery of Carmustine 4-Hydroperoxycyclophosphamide and Paclitaxel From a Biodegradable Polymer Implant in the Monkey Brain, Cancer Research 58; 672-684:1998.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide Fibulin-5 (the skin tissue) levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect, the Fibulin-5 is comprised in an effective amount in a cosmetic composition.

The cosmetic composition of this invention comprises not only the Fibulin-5 but also ingredients conventionally used in cosmetic compositions such as auxiliaries including stabilizers, solubilizers, Vitamins, colorants and flavors, and carriers.

The cosmetic compositions of this invention may be formulated in a wide variety of forms, for example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these substances.

In the formulation of powder or spray, it may comprise lactose, talc, silica, aluminum hydroxide, calcium silicate, polyamide powder and mixtures of these substances. Spray may additionally comprise the customary propellants, for example, chlorofluorohydrocarbons, propane/butane or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butylglycol, oils, in particular cottonseed oil, groundnut oil, maize germ oil, olive oil, castor oil and sesame seed oil, glycerol fatty esters, polyethylene glycol and fatty acid esters of sorbitan or mixtures of these substances.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these substances.

The formulation of soap may comprise alkali metal salts of fatty acids, salts of fatty acid hemiesters, fatty acid protein hydrolyzates, isethionates, lanolin, fatty alcohol, vegetable oil, glycerol, sugars or mixtures of these substances.

The formulation of a surfactant-containing cleanser may comprise as carriers aliphatic alcohol sulfate, aliphatic alcohol ether sulfate, sulfosuccinic acid monoester, isethionate, imidazoliniurn derivatives, methyltaurate, sarcosinate, fatty acid amide ether sulfate, alkylamidobetaine, aliphatic alcohols, fatty acid glycerides, fatty acid diethanolamide, plant oils, lanolin derivatives or ethoxylated glycerol fatty acid ester.

As used herein the term “about” refers to ±10% .

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Tissue Specimens

Six patients, who underwent surgical treatment for de novo keloid scars, enrolled in this study. The study was approved by the Rambam Hospital Institutional Review Board.

Keloids were diagnosed on the basis of clinical appearance, history and anatomical location. Diagnoses were confirmed by histological examinations with Hematoxylin/Eosin (H&E), Masson-Trichrome, and Reticulin (Sigma, USA) staining.

Histological Studies

Keloid tissues were collected during surgical excision. For histology analysis, parts of the tissue samples were fixed in 10% formaldehyde for 24 hours, embedded in paraffin blocks, and sectioned to 5 μm thickness with LEICA RM2255 microtome.

Isolation of Keloid-Derived FLCs

Tissue samples were transferred to the laboratory in test tubes containing 50 ml tissue growth medium, which comprised DMEM, penicillin (1/100), streptomycin (1/100), HEPES (1/50), 20% FBS (Biological Industries, Israel) and GlutaMAX (1/1000 μl, Invitrogen), maintained at 4° C. Tissues were washed six times in phosphate-buffered saline (PBS, Biological Industries, Israel). Small pieces (<5 mm) were incubated on petri dishes (Greiner, Germany) or tissue culture plates (Greiner, Germany) with the medium detailed above, at 37° C., 5% CO₂. Every 48 hr, the growth medium was replaced with a fresh one. When fibroblast migration was observed, the cells were collected by trypsinization. The first cells to grow out from the scar were called passage 1.

Characterization of the Keloid-Derived FLCs

Chemical stains were used for characterizing the keloid-derived extensions. For DAPI (4,6-diamidino-2-phenylindole, Santa Cruz Biotechnology, USA) staining, cells were incubated in 6-well plates at 37° C., 5% CO₂ for 48 hr. Cells were fixed in 4% paraformaldehyde for 30 min, washed with PBS and stained with DAPI for 10 min. Cells were observed by Nikon eclipse TS100 fluorescent microscopy at 340 nm wavelength.

For protein staining, cells were incubated in 6-well plates at 37° C., 5% CO₂ for 48 hr, and then fixed in 4% paraformaldehyde for 30 min and washed with PBS. The fixed cells were stained with coomassie blue (Sigma, USA). Fibulin-5 expression levels in the scar explants were determined by immunohistochemistry.

Keloid-Derived FLCs—Proliferation Assay

Ninety six-well plates were coated with 100 μl gelatin 0.1% w/v or 400 ng fibulin-5 (MGVS, Israel produced in CHO cells by a retroviral vector pLXSN-UP50), at 4° C. overnight. Keloid-derived FLCs (2×10³/well) were incubated, with five repetitions for each coating, with 100 μl growth medium, DMEM+2% FBS. After 48 hr, the medium was replaced by a fresh one. Cells were incubated for 72 hrs to 96 hrs according to cell confluence, at 37° C., 5% CO₂. At the end of the incubation period, 50 μl XTT reagent (Biological Industries, Israel) were added to the cells. Then, the cells were incubated for an additional 3 hr at 37° C., 5% CO₂, in total darkness. The plate was read using the ELISA reader (Tecan sunrise ATR F039300) at 492/640 nm wavelengths. Cells at passage 3-5 were used for these experiments.

Keloid-Derived FLCs—Adhesion Tests

Keloid-derived FLCs (2×10⁴/well) were incubated in 6-well plates coated with gelatin or 40 μl fibulin-5; and washed with DMEM+FBS, as described above. The wells were washed three times with PBS at 20,40,60,90,120,180 and 240 minutes; positive control wells were left unwashed. After each wash a fresh growth medium was added to the wells. After 240 minutes, cells were prepared and analyzed as described above.

To examine the role of integrin beta-1, keloid-derived FLCs were coated with fibulin-5 and incubated, as described above, with or without the addition of 20 μg/106 more FLCs and anti-human integrin beta-1(CD29 MAB 17781 R&D Systems) antibody, to block the activity of integrin beta-1. The wells were washed with PBS at 90,120, and180 minutes. At the end of each wash, a fresh growth medium was added to each well. After 180 minutes, cells were prepared and analyzed as described above. For adhesion experiments cells at passage 4 were used.

Statistical Analysis

Cell estimation was measured in triplicate or quintuplicate and the mean±SD was calculated. The significance of the difference between the groups was analyzed statistically by a Student's t-test to compare the mean cell numbers and optical density. The difference between the means for all conditions was considered statistically significant at P<0.05.

### Example 2

### Characteristics of Keloid Explants & Keloid-Derived FLCs

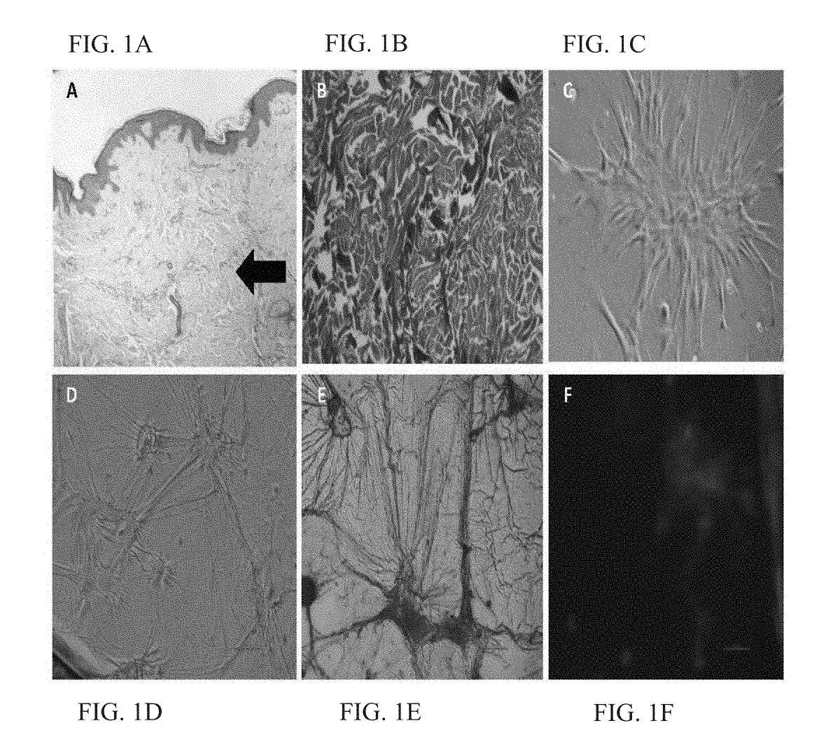

Table 1 presents characteristics of the study participants and of the keloids. The primary FLC cell lines used in the reported experiments appear in the left column of the Table. FIG. 1A-1F depict the large, broad, stretched and closely arranged collagen bundles (FIG. 1A). Staining with Masson's Trichrome (FIG. 1B) and H&E verified the identity of collagen bundles, with a spindle shape morphology that migrated out of the scar tissues (FIG. 1C). In two primary cell lines of FLC (E & F, Table 1) stretching structures surrounding the growing cells were noticed (FIG. 1D). These structures appeared as elongated extensions stretching from one FLC body to another. The structures showed an obvious staining with coomassie blue and not with DAPI, indicating a proteinaceous composition (FIGS. 1D-F). Immunochemistry testing demonstrated that fibulin-5 expression was negligible in the scar explants (Data not shown).

TABLE 1
Characteristics of the participants and
the scar tissues included in the study

| Human keloid | Pa- | Patient | | Keloid | Keloid |
|---|---|---|---|---|---|
| fibroblast-like | tient | age | | length | weight |
| cell (FLC) lines | sex | (year) | Keloid location | (cm) | (gr) |
| A | F | 42 | Upper back | 3.6 | 5.69 |
| B | M | 57 | Right shoulder | 3 × 1.3 | 1.84 |
| C | F | 44 | Ear | 1.5 × 1 | 1.23 |
| D | F | 21 | Ear | N/A | 1.22 |
| E | F | 50 | Left shoulder | 5 × 2.5 | 7.85 |
| F | F | 28 | Upper back | 2 × 3 | 4.81 |

### Example 3

### Fibulin-5 Inhibits the Proliferation of Keloid-Derived FLCs

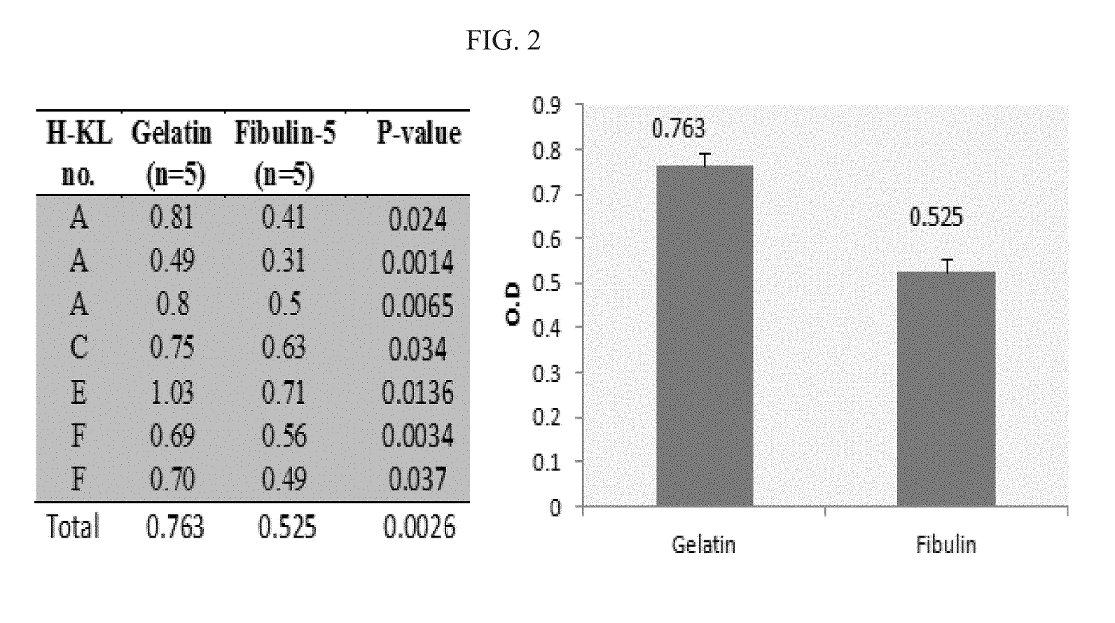

FLCs from different patients incubated with gelatin coating, showed different proliferation rates for the different FLC lines (Data not shown). To evaluate the effect of fibulin-5 on cell-proliferation, FLCs of different cell lines were seeded onto pre-coated dishes with fibulin-5 or gelatin, and the proliferation rates were assayed for 72-96 hrs. A significant reduction (p<0.05) in proliferation rates was found on all tested FLC-lines, indicating that fibulin-5 coating significantly reduces FLC proliferation during 72-96 hrs, compared to gelatin (FIG. 2).

### Example 4

### Fibulin-5 Promotes Adhesion of Keloid-Derived FLCs Through Binding to Integrin Beta-1

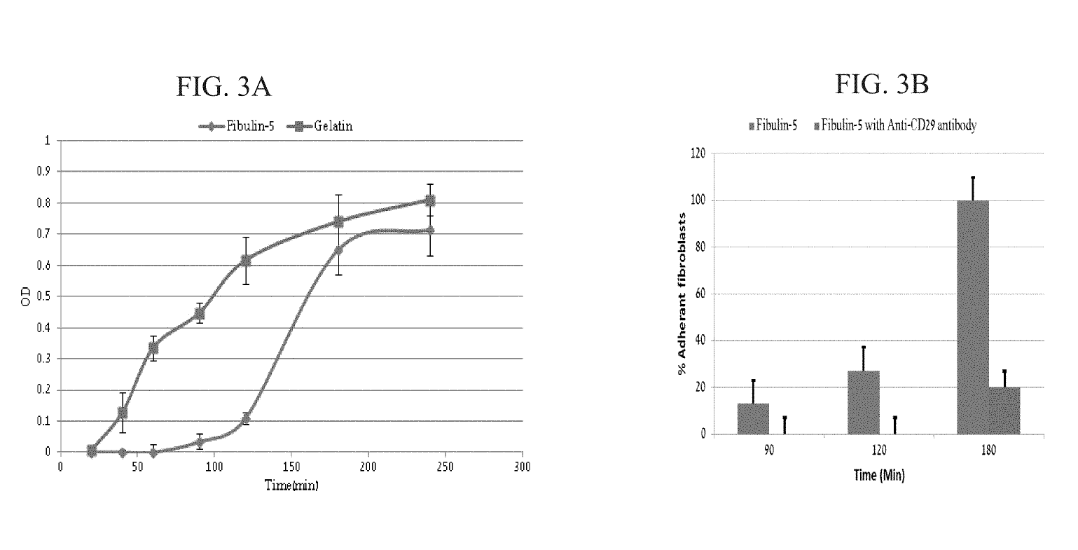

To evaluate the rate and mechanism by which fibulin-5 interacts with FLCs, the adhesion rate of these cells was tested. FLCs from the F cell line were used since they demonstrated the highest proliferation rate. When plated on plates pre-coated with gelatin, the FLCs displayed a linear attachment rate; cells attached to the gelatin-coated surface reached full adhesion after 180 minutes (FIGS. 3A-3B). In contrast, FLCs seeded onto wells pre-coated with fibulin-5 showed delayed adhesion. No adhesion was observed in the first 90 minutes; adhesion subsequently occurred, until the same plateau as gelatin was reached at 180 minutes (FIGS. 3A-3B).

To examine the role of integrin beta-1 in FLC-fibulin-5 interactions, an anti-human CD29 antibody (anti-human integrin beta-1) was added to the fibulin-5 medium. Inhibition of integrin beta-1 resulted in significant reduction of adherent cells to the fibulin-5 pre-coated wells (FIGS. 3A-3B). Adherent FLCs, seeded on wells with CD29 antibody, were first detected only after 180 minutes, compared to the detection, at 90 minutes, of a significant number of cells seeded on fibulin-5 alone.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited in the Application

1. Tuan T, Nichter L. The molecular basis of keloid and hypertrophic scar formation. Mol Med Today 1998; 4:19-24.
2. Atiyeh B, Costagliola M, Hayek S, Keloid or hypertrophic scar: the controversy: review of the literature. Ann Plast Surg 2005; 54:676-680.
3. Butler P, Longaker M, Yang G, Current progress in keloid research and treatment. J Am Coll Surg 2008; 206:731-741.
4. Bran G, Goessler U, Hormann K, et al. Keloids: current concepts of pathogenesis (review). Int J Mol Med 2009; 24:283-293.
5. Friedman D, Boyd C, Mackenzie J, et al. Regulation of collagen gene expression in keloids and hypertrophic scars. J Surg Res 1993; 55:214-222.
6. Syed F, Ahmadi E, Iqbal S, et al. Fibroblasts from the growing margin of keloid scars produce higher levels of collagen I and III compared with intralesional and extralesional sites: clinical implications for lesional site-directed therapy. Br J Dermatol 2011; 164:83-96.
7. Shah M, Forema D, Ferguson M. Control of scarring in adult wounds by neutralising antibody to transforming growth factor beta. The Lancet 1992; 339:213-214.
8. Yoshimoto H, Ishihara H, Ohtsuru A, et al. Overexpression of insulin-like growth factor-1(IGF-I) receptor and the invasiveness of cultured keloid fibroblasts. Am Journal Pathol 1999; 154:883-889.
9. Nirodi C, Devalaraja R, Nanney L, et al. Chemokine and chemokine receptor expression in keloid and normal fibroblasts. Wound Repair and Regeneration 2000; 8:371-382.
10. Tanaka A, Hatoko M, Tada H, et al. Expression of p53 family in scars. J Dermatol Sci 2004; 34:17-24.
11. Nakamura T, Ruiz-Lozano P, Lindner V, et al. DANCE, a novel secreted RGD protein expressed in developing, atherosclerotic, and balloon-injured arteries. J Biol Chem 1999; 274:476-483.
12. Yanagisawa H, Schluterman M, Brekken R. Fibulin-5, an integrin-binding matricellular protein: its function in development and disease. J Cell Commun Signal 2009; 3:337-347.
13. Schiemann W, Blobe G, Kalume D, et al. Context-specific effects of fibulin-5(DANCE/EVEC) on cell proliferation, motility and invasion. Fibulin-5 is induced by transforming growth factor-beta and affects protein kinase cascades, J Biol Chem 2002; 277:27367-27377.
14. Preis M, Cohen T, Sarnatzki Y, et al. Effects of fibulin-5 on attachment, adhesion, and proliferation of primary human endothelial cells. Biochem Biophys Res Commun 2006; 348:1024-1033.
15. Lomas A, Mellody K, Freeman L, et al. Fibulin-5 binds human smooth-muscle cells through alpha5beta1 and alpha4beta1 integrins, but does not support receptor activation. Biochem. J 2007; 405:417-428.
16. Lee M, Roy N, Mogford J, et al. Fibulin-5 promotes wound healing in vivo. J Am Coll Surg 2004; 199:403-410.
17. Yanagisawa H, Davis E, Starcher B, et al. Fibulin-5 is an elastin-binding protein essential for elastic fibre development in vivo. Nature 2002; 415:168-171.
18. Nakamura T, Lozano P, Ikeda Y, et al. Fibulin-5/DANCE is essential for elastogenesis in vivo Nature 2002; 415:171-175.
19. Kamath N, Ormsby A, Bergfeld W, House N. A light microscopic and immunohistochemical evaluation of scars. J Cutan Pathol 2002; 29:27-32.
20. Ikeda M, Naitoh M, Kubota H, et al. Elastic fiber assembly is disrupted by excessive accumulation of chondroitin sulfate in the human dermal fibrotic disease, keloid. Biochem Biophys Res Commun 2009; 390:1221-1228.
21. Wang Z, Fong K, Phan T, et al. Increased transcriptional response to mechanical strain in keloid fibroblasts due to increased focal adhesion complex formation. J Cell Phys 2006; 206:510-517.
22. Babu M, Diegelmann R, Oliver N. Fibronectin is overproduced by keloid fibroblasts during abnormal wound healing. Mol Cell Biol 1989; 9:1642-1650.

1. A method of treating a keloid scar or a medical condition associated therewith, the method comprising administering to a subject in need thereof a therapeutically effective amount of fibulin-5, thereby treating the keloid scar.

2-3. (canceled)

4. A method of inhibiting proliferation and/or adherence of fibroblast-like cells from a keloid scar, the method comprising contacting the fibroblast-like cells with an effective amount of fibulin-5, thereby inhibiting proliferation and/or adherence of fibroblast-like cells from the keloid scar.

5. The method of claim 4, wherein said contacting is effected in-vivo.

6. The method of claim 4, wherein said contacting is effected ex-vivo or in-vitro.

7. The method of claim 4, wherein said fibroblast-like cells are comprised in a tissue.

8. The method of claim 4, wherein said fibroblast-like cells are primary cells.

9. The method of claim 4, wherein said fibroblast-like cells are a cell line.

10. A cosmetic composition comprising an effective amount of fibulin-5 and a cosmetically acceptable carrier.

11. The method of claim 1, wherein said keloid scar is caused by external injuries.

12. The method of claim 1, wherein said keloid scar is caused by surgical procedures.

13. The method of claim 1, wherein said medical condition is selected from the group consisting of pain, inflammation and vascularization.

14. The method of claim 1, wherein said administering is effected locally.

15. The method of claim 14, wherein said administering is effected epicutaneously, subcutaneously or intradermally.

16. The method of claim 1, wherein said fibulin-5 comprises an integrin beta binding domain.

17. The method of claim 16, wherein said fibulin-5 is full length fibulin-5.

18. The method of claim 1, wherein said fibulin-5 is human fibulin-5.


SEQUENCE LISTING (2 sequences)

SEQ ID NO: 1 - protein, 448 aa, Homo sapiens
MPGIKRILTVTILALCLPSPGNAQAQCTNGFDLDRQSGQCLDIDECRTIPEACRGDMMCVNQNGGYLCIPRTNPVYRGPYSNPYSTPYSGPYPAAAPPLSAPNYPTISRPLICRFGYQMDESNQCVDVDECATDSHQCNPTQICINTEGGYTCSCTDGYWLLEGQCLDIDECRYGYCQQLCANVPGSYSCTCNPGFTLNEDGRSCQDVNECATENPCVQTCVNTYGSFICRCDPGYELEEDGVHCSDMDECSFSEFLCQHECVNQPGTYFCSCPPGYILLDDNRSCQDINECEHRNHTCNLQQTCYNLQGGFKCIDPIRCEEPYLRISDNRCMCPAENPGCRDQPFTILYRDMDVVSGRSVPADIFQMQATTRYPGAYYIFQIKSGNEGREFYMRQTGPISATLVMTRPIKGPREIQLDLEMITVNTVINFRGSSVIRLRIYVSQYPF

SEQ ID NO: 2 - DNA, 1347 bp, Homo sapiens
atgccaggaataaaaaggatactcactgttaccattctggctctctgtcttccaagccctgggaatgcacaggcacagtgcacgaatggctttgacctggatcgccagtcaggacagtgtttagatattgatgaatgccgaaccatccccgaggcctgccgaggagacatgatgtgtgttaaccaaaatggcgggtatttatgcattccccggacaaaccctgtgtatcgagggccctactcgaacccctactcgaccccctactcaggtccgtacccagcagctgccccaccactctcagctccaaactatcccacgatctccaggcctcttatatgccgctttggataccagatggatgaaagcaaccaatgtgtggatgtggacgagtgtgcaacagattcccaccagtgcaaccccacccagatctgcatcaatactgaaggcgggtacacctgctcctgcaccgacggatattggcttctggaaggccagtgcttagacattgatgaatgtcgctatggttactgccagcagctctgtgcgaatgttcctggatcctattcttgtacatgcaaccctggttttaccctcaatgaggatggaaggtcttgccaagatgtgaacgagtgtgccaccgagaacccctgcgtgcaaacctgcgtcaacacctacggctctttcatctgccgctgtgacccaggatatgaacttgaggaagatggcgttcattgcagtgatatggacgagtgcagcttctctgagttcctctgccaacatgagtgtgtgaaccagcccggcacatacttctgctcctgccctccaggctacatcctgctggatgacaaccgaagctgccaagacatcaacgaatgtgagcacaggaaccacacgtgcaacctgcagcagacgtgctacaatttacaagggggcttcaaatgcattgaccccatccgctgtgaggagccttatctgaggatcagtgataaccgctgtatgtgtcctgctgagaaccctggctgcagagaccagccctttaccatcttgtaccgggacatggacgtggtgtcaggacgctccgttcccgctgacatcttccaaatgcaagccacgacccgctaccctggggcctattacattttccagatcaaatctgggaatgagggcagagaattttacatgcggcaaacgggccccatcagtgccaccctggtgatgacacgccccatcaaagggccccgggaaatccagctggacttggaaatgatcactgtcaacactgtcatcaacttcagaggcagctccgtgatccgactgcggatatatgtgtcgcagtacccattctga